(19) United States
(12) Patent Application Publication
(10) Pub. No.: US 2002/0172780 A1
(43) Pub. Date: Nov. 21, 2002

(54) Method and apparatus for treating surfaces with a plasma generated by electron cyclotron resonance

(76) Inventor: Ward Dean Halverson, Cambridge, MA (US)

Correspondence Address: NUTTER MCCLENNEN & FISH LLP, WORLD TRADE CENTER WEST, 155 SEAPORT BOULEVARD, BOSTON, MA, 02210-2604, US

(21) Appl. No.: 09/849,065

(22) Filed: May 4, 2001

(51) Int. Cl. H05H 1/24; A61L 2/00; C23F 1/00; C23C 16/00

(52) U.S. Cl. 427/569; 427/571; 427/2.1; 118/723 MR; 118/723 MA; 156/345.42

(57) ABSTRACT

The present invention provides methods and apparatus for plasma treatment of tubing surfaces. In one aspect, the invention provides a method for treating a tubing surface which calls for generating a gaseous plasma within a spatially localized region of space by electron cyclotron resonance (ECR), and exposing the surface to this plasma for a selected time period to treat the surface. Subsequent to the plasma treatment, the treated surface can be optionally coated with a selected compound, such as a bioactive material, e.g., anti-biotic or anti-coagulants.

### BACKGROUND OF THE INVENTION

The present invention relates generally to methods and apparatus for treating surfaces, and more particularly, to methods and apparatus for plasma treatment of surfaces.

A variety of chemical, biochemical, and physical techniques are known for treating surfaces, e.g., polymer surfaces. For example, ionizing radiation, e.g., ultraviolet radiation, or ionic plasma can be utilized to provide sterilization, cross-linking or bond scission on polymer surfaces. Further, ion implantation and plasma treatment techniques are employed in various clinical applications, such as treating exterior surfaces of catheters formed from silicone rubber to reduce surface friction and to increase surface hydrophilicity.

Plasma treatment techniques are typically better suited for modifying polymer surfaces than many other surface modification techniques. For example, plasma treatment techniques permit deposition of organic or inorganic coatings onto a polymer surface directly from the plasma. Further, the plasma conditions can be varied to provide different treatments of a polymer surface. For example, variations of discharge power, frequency, pressure, gas species, and plasma geometry can modulate the effect of the plasma on a polymer surface.

Conventional discharges, e.g., glow or corona discharges, are not suitable for localized treatment of a surface. That is, such conventional discharges do not typically permit treating a selected portion of the surface of an article without affecting the remainder of the surface.

Thus, a need exists for providing techniques for plasma treatment of tubing that allow treating both the internal and the external surfaces of a tubing. Further, a need exists for providing plasma treatment techniques that allow selective and localized treatment of a surface.

### SUMMARY OF THE INVENTION

The present invention provides methods and apparatus for plasma treatment of surfaces, such as internal and/or external surfaces of non-conductive tubular articles, such as catheter lines, cannulae and other medical instruments with lumens. In one aspect, the invention provides a method of treating a tubing surface which calls for generating a gaseous plasma within a spatially localized region of space by electron cyclotron resonance (ECR), and exposing at least a portion of the surface of the tubing to the plasma for a selected time to treat the surface. The treated surface can be either the interior wall, i.e., the lumen, of the tubing or it can be its exterior surface or both.

In a related aspect, the gaseous plasma can be generated by providing a static magnetic field having a selected strength, i.e., amplitude (B), within a region of space in which a quantity of gas, e.g., argon, is contained, or through which the gas is flowing. The gas is then irradiated with electromagnetic radiation having a frequency which is substantially equal to electron cyclotron resonance frequency (fc) at the applied magnetic field strength. The radiation causes the gas to ionize, thus producing a gaseous plasma.

The ECR frequency is related to the amplitude of the applied field by the following equation:

$${f}_{c} = \frac{1}{2 π} e B / m . \qquad Eq . ( 1 )$$

where f_{c} denotes the ECR frequency, B denotes the amplitude of the magnetic field, and e and m denote the charge and the mass of an electron, respectively.

In general, any combination of the radiation frequency and magnetic field amplitude that substantially satisfies the constraint imposed by Equation (1) can be utilized to obtain an ECR generated plasma in accord with the teachings of the invention. Thus, various radiation frequencies and magnetic field strengths can be utilized to create an ECR generated plasma in accord with the teachings of the invention. The radiation frequency can be selected to be in a range of about 1 GHz to about 15 GHz, and the applied static magnetic field can be selected to have an amplitude in a range of approximately 300 Gauss to approximately 5500 Gauss. For each selected radiation frequency, the amplitude of the magnetic field is chosen in accord with the resonance condition delineated in the above Equation (1). For example, in one embodiment of the invention, the frequency of the electromagnetic radiation is about 2.45 GHz, and the amplitude of the applied magnetic field is approximately 875 Gauss. Alternatively, the frequency of the electromagnetic radiation can be selected to be about 10 GHz when the amplitude of the applied magnetic field is approximately 3571 Gauss.

An ECR plasma of the invention can be generated and maintained in a gas having a wide range of pressures. For example, the gas pressure can be in a range of about 0.1 Pa to about 1000 Pa. The gas pressure is preferably in a range of about 1 Pa to about 10 Pa. More preferably, the gas pressure is selected to be approximately 5 Pa. Further, a variety of different gases can be employed to practice the invention. These gases can include, but are not limited to, noble gases, such as argon, diatomic gases, such as oxygen and nitrogen, hydrocarbons, such as methane and butane, and fluorinated hydrocarbons, such as tetrafluoromethane. Moreover, various mixtures of different gases can be utilized to create an ECR plasma in accord with the teachings of the invention. For example, a mixture of argon and oxygen (e.g., a mixture having 50% molar concentration of argon and 50% molar concentration of oxygen) or a mixture of argon and ammonia can be utilized to practice the invention.

The method of the invention can be utilized to treat the surfaces of tubes having a wide range of sizes. For example, hollow instruments having inner diameters in a range of about 0.5 mm to about 20 mm can be treated by a plasma produced in accord with the teachings of the invention. The invention is particularly useful in forming coatings on the inner surfaces of very narrow lumens.

In another aspect, the invention provides a processing method in which a tube is passed through the plasma zone, i.e., the region of space in which an ECR-generated gaseous plasma is present, at a selected rate to expose different portions of the inner and/or outer walls of the tube to the plasma. The tube can be drawn through the plasma zone, for example, at a uniform rate to ensure uniform treatment of its entire inner or outer walls. Alternatively, an object undergoing treatment can be drawn through the zone at a variable speed to provide a graded treatment effect.

In a related aspect, subsequent to the plasma treatment, the surface of the tubing can be optionally coated with a selected material, e.g., by dipping or introducing a reagent solution into the lumen. The coating material can be inorganic or organic, e.g., an organic polymer. In some embodiments, the coating material provides a bioactive coating over the treated surface. For example, anti-coagulants, anti-thrombotics, antibiotics, anti-microbials, and other biologically and/or therapeutically active compounds can be applied to an inner or outer wall of a tubing, such as a catheter, after it has been treated with an ECR plasma in accord with the teachings of the invention. One exemplary coating agent is heparin, a compound with both anti-coagulant and anti-thrombotic properties. In addition, the coating material can include one or more proteins, enzymes, vitamins, and/or minerals. Moreover, the coating material can be selected such that it provides anti-inflammatory analgesic properties. In some embodiments, the coating material is selected to have cell growth properties, i.e., it allows growing cells on the coating surface.

The methods of the present invention allow coating both the external and the internal surfaces of a tubular article, e.g., a catheter. For example, anti-biotic, anti-microbial, or anti-inflammatory materials can be utilized to coat an external surface of a tubular article that has been treated by exposure to an ECR-generated plasma in accord with the teachings of the present invention.

In a related aspect, the methods of the invention can be utilized to deposit a selected coating onto a surface directly from an ECR-generated plasma. For example, an ECR plasma generated in a methane or butane gas can be utilized to directly deposit carbon containing materials onto a surface exposed to the plasma.

Another aspect of the invention relates to providing apparatus for implementing the method of the invention described above. One such apparatus can include a permanent magnet that produces a static magnetic field within a region of space with a selected strength, i.e., amplitude. The apparatus can further include a conduit that can receive gas from a source and deliver a selected quantity or flow of the gas into a section of the tubing, e.g., a section of the lumen of the tubing, which is placed within this region of space. An electromagnetic energy generator is positioned so as to irradiate the region of space in which the magnetic field is present. The electromagnetic radiation has a frequency that is substantially equal to the electron cyclotron frequency at the magnetic field amplitude so as to ionize the gas and create a plasma, i.e., to generate a plasma zone. The section of the tubing present in the plasma zone is treated through exposure to the plasma.

The electromagnetic energy generator can be, for example, a magnetron or an IMPATT diode. The apparatus can also include a radiation guidance device, such as a waveguide or a coaxial cable, to transmit the electromagnetic radiation from the radiation source to the region of space in which a gaseous plasma is created. In addition, in certain microwave frequency ranges, a radiation coupling device, e.g., a horn, can be connected to the radiation guidance device to provide an impedance match between the guidance device and free space, thus enhancing the coupling of radiation into the region of space in which the gaseous plasma is generated.

Another apparatus for implementing the method of the invention can include a magnet for producing a static magnetic field having a pre-defined amplitude within a selected region of space. The apparatus can further include a waveguide section that can be connected to a source of radiation. An enclosure for storing a gas, e.g., argon, can be created adjacent to or within the waveguide. Radiation can be transmitted into the enclosure by employing, for example, a window positioned within the waveguide. The window is preferably transparent to microwave radiation to allow coupling of radiation from the source into the gas in the enclosure. A port, which is preferably vacuum tight, allows insertion of at least a portion of a tube into the gas enclosure. The radiation frequency is selected to be substantially equal to the ECR frequency at the pre-defined magnetic field amplitude so as to ionize the gas within the enclosure and hence create a gaseous plasma zone. The exposure of the surface of the tube to this plasma for a selected time period results in plasma treatment of the surface.

Illustrative embodiments of the invention are described below with reference to the following drawings.

### BRIEF DESCRIPTION OF THE DRAWINGS

FIG. 1 is a flow chart depicting various steps performed by an exemplary embodiment of a method according to the teachings of the invention for treating tubing surfaces,

FIG. 2 illustrates an exemplary apparatus for treating a tubular surface by an ECR-generated plasma in accord with the teachings of the invention,

FIG. 3 illustrates another exemplary apparatus for treating the lumen of a tubing by exposing it to an ECR-generated plasma in accord with the teachings of the invention, and

FIG. 4 illustrates still another exemplary apparatus according to the invention for treating an external surface of a tubing by exposing it to an ECR-generated plasma.

### DETAILED DESCRIPTION

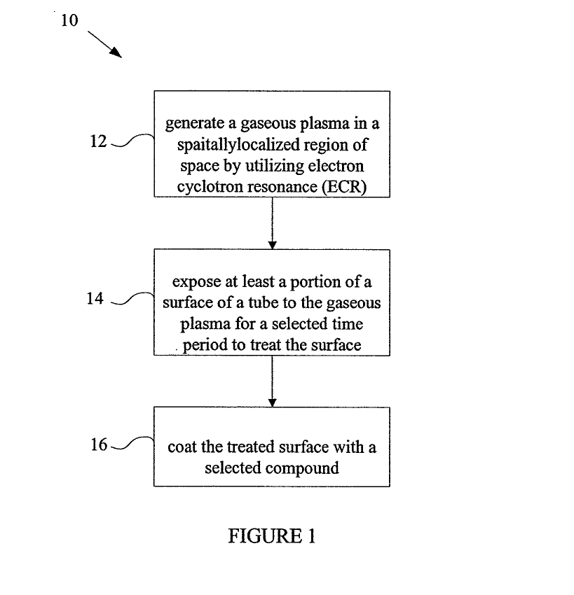

The present invention provides methods and apparatus for treating interior and/or exterior surfaces of tubular article, e.g., polymer tubing, by exposing the surface to a spatially localized gaseous plasma. More particularly, FIG. 1 depicts a flow chart 10 illustrating various steps according to one embodiment of the invention in which, in step 12, a gaseous plasma discharge is generated within a spatially localized region of space by utilizing electron cyclotron resonance (ECR). One embodiment generates the gaseous plasma by providing a static magnetic field having a selected amplitude in a region of space in which a quantity of a gas, e.g., argon, is contained, or through which the gas is flowing. Electromagnetic radiation having a frequency substantially equal to the ECR frequency at the magnetic field amplitude irradiates the gas in this region of space so as to ionize it and create a plasma zone. For example, microwave radiation at a frequency of about 2.45 GHz and a static magnetic field having an amplitude of approximately 875 Gauss can be utilized to create a spatially-localized plasma in a gas having a pressure in a range of about 0.1 Pa to about 1000 Pa.

A ECR-generated plasma is qualitatively and quantitatively distinct from a DC or a low-frequency RF-heated discharge. It is understood that electrons in an ECR plasma are accelerated by the electric field of an applied electromagnetic radiation, e.g., microwave radiation having a frequency of about 2.45 GHz, and are forced simultaneously to gyrate in circular orbits by the applied magnetic field. A resonance condition is achieved when the gyration frequency and the frequency of the electromagnetic radiation are substantially equal. In other words, a resonance condition is achieved when the frequency of the applied electromagnetic radiation is substantially equal to the electron cyclotron frequency (f_{c}) given by the above Equation (1).

Under ECR conditions, electrons can gain sufficient energy to ionize the gas. Initially, a few free electrons are accelerated to energies high enough to ionize some neutral atoms of the gas. The electrons generated from the ionizing collisions are then accelerated to energies sufficient for ionizing other neutral atoms, and hence a steady-state plasma is created and maintained.

In step 14, a portion of a surface of the tubing is exposed to the ECR-generated plasma for a selected time period to treat the surface. This plasma treatment can cause specific, controllable, and spatially localized modification of the surface. For example, the plasma treatment can increase the surface energy, and hence the wettability of the surface. When the surface is formed of a polymer, the plasma treatment can cross-link the polymer to improve its mechanical and/or chemical properties. Further, the plasma treatment can break chemical bonds (scission) to increase chemical reactivity, smooth the surface, seal pores, reduce friction, clean and/or sterilize the surface.

In one embodiment of the invention, a surface to be treated is exposed to an ECR-generated plasma for a time period ranging from about one second to about one minute. More preferably, the exposure time is a few seconds, e.g., 10 seconds. Different exposure times can be selected for causing different modifications of a surface. For example, a short exposure time, e.g., one second, may be sufficient to activate a surface whereas a longer exposure time, e.g., a few seconds, may be needed for causing more severe changes, e.g., bond scission.

With continued reference to FIG. 1, in step 16, the treated surface can be optionally coated with a selected compound. For example, after treating the interior surface of a catheter by exposing it to an ECR plasma according to the teachings of the invention, a coating of a bioactive material can be applied to the surface by utilizing any of the known methods in the art, e.g., flowing an aqueous solution of the bioactive material through the treated tubing. Such a bioactive material can include, but is not limited to, antibiotics, anti-coagulants, such as heparin, anti-thrombogenic or other therapeutic compounds.

The method of the invention can be utilized to treat both the internal and external surfaces of a tubing. For example, to treat the internal wall of a tubing, an embodiment of the invention places at least a portion of the tubing in the ECR zone, and causes the flow of a low pressure gas (on the order of a few pascals), such as argon, through this portion. The gas breaks down to form a plasma within the ECR zone, and extinguishes outside the ECR zone. Further, the formation of plasma in the proximity of the external wall of the tubing is prevented by maintaining the external atmosphere at either a high enough or a low enough pressure so as to inhibit gas breakdown. The tubing can be drawn through the ECR zone to treat different sections of the internal wall.

Another embodiment of the invention generates an ECR plasma only outside a tubing to treat its external wall. In such an embodiment, the internal gas breakdown can be prevented, for example, by sealing, pressurizing or evacuating the lumen of the tubing. For example, pressurizing the lumen to a pressure of about one atmosphere with purified air or oxygen can be sufficient to prevent plasma formation. Alternatively, maintaining a vacuum of less than about 10⁻⁴ pascal (7.5×10⁻⁷ torr) can prevent formation of a plasma.

The method of the invention can advantageously generate and maintain a plasma over a wide range of parameters, e.g., gas pressure, molecular and atomic species present in the gas, to provide specific, selective and spatially-localized treatment of both internal and external walls of electrically non-conductive tubing. In addition, the method of the invention allows fine tuning the process parameters, e.g., gas pressure, to obtain different conditions on interior and exterior surfaces of a tube. For example, as discussed above, the method of the invention can be utilized to expose the interior wall of a tube to an ECR generated plasma while the exterior surface remains unaffected. Further, as discussed in more detail below, the method of the invention can be implemented such that many tubes are treated simultaneously, thus providing a cost effective surface treatment process for small, medium, or large-scale industrial production.

The method of the invention has additional advantages in that an ECR plasma can be generated and maintained over a wider range of discharge parameters, e.g., pressure, microwave power, atomic or molecular species of the gas, than conventional glow or corona discharge plasmas. Thus, the method of the invention can provide a wider range of conditions for plasma treatment of surfaces, e.g., polymer tubing, than those provided by conventional plasma treatment techniques.

The method of the invention finds a variety of applications. For example, it can be utilized to treat surfaces of medical catheters, and particularly, the interior walls of such catheters. An ECR-generated plasma of the invention can provide a uniform treatment of the interior wall of such catheters, or other non-conductive tubing, as the catheters move through the resonance region. In addition, the plasma treatment can cause removal of particulates or adsorbed liquid contaminants from the internal wall. It can further provide pore sealing, coating, sterilization, or surface activation to promote adhesion of a bioactive, e.g., antithombotic or anibiotic, coating to the surface.

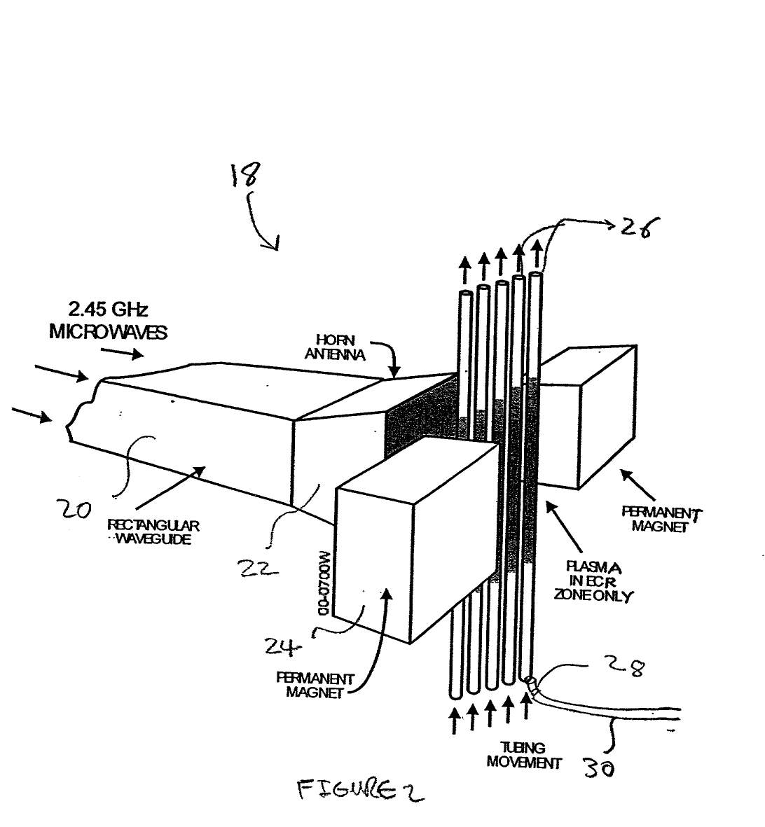

FIG. 2 illustrates an exemplary apparatus 18 for treating the lumen of tubing by exposure to an ECR-generated plasma in accord with the teachings of the invention. The apparatus 18 includes a radiation guiding device 20, such as a waveguide, for transmitting radiation from a radiation source (not shown), such as a microwave oscillator, to a region of space in which an ECR plasma is generated (ECR zone).

In the exemplary apparatus 18, a radiation coupling device 22, such as a horn, connected to the waveguide 20 improves impedance match between the waveguide and the outside environment, and thereby enhances the degree of coupling of radiation from the waveguide to the outside environment. A radiation frequency in a range of about 1 to 15 GHz can be utilized to practice the present invention. In this exemplary embodiment, the radiation source provides microwave radiation at a frequency of about 2.45 GHz.

Microwave power levels in a range of about 10 Watts to 500 Watts can be utilized to generate an ECR plasma in accord with the teachings of the invention. The power level is preferably in a range of about 75 Watts to 150 Watts. More preferably, the power level is selected to be approximately 100 Watts.

The microwave radiation source can be, for example, a magnetron or an IMPATT diode. Those skilled in the art will appreciate that radiation frequencies that can be utilized for practicing the invention are not limited to 2.45 GHz, which is provided herein only for illustrative purposes. Further, the radiation guiding device is not limited to waveguide. For example, a coaxial cable can be utilized in some frequency ranges to transmit radiation from a source to the plasma zone.

A permanent magnet 24 creates a magnetic field in the ECR region having an amplitude that is related to the frequency of the radiation source by the above Equation (1). The direction of the magnetic field vector of the magnet 24 is preferably selected to be substantially perpendicular to the polarization of the radiation field provided by the radiation source.

A plurality of tubes 26, formed of an electrically non-conductive material, e.g., polymer medical catheters, can be positioned such that at least a portion of each tube resides within the ECR zone. Further, the apparatus 18 can provide each tube with couplings, such as coupling 28, that connects each tube to a conduit 30 adapted to couple to a source of gas to deliver a selected quantity, or flow, of gas to the lumen of each tube. The quantity and/or the flow rate of gas delivered to each tube is controlled such that the pressure within the lumen of each tube, particularly in the ECR zone, lies within a range of about 0.1 Pascal to about 1000 Pascal. Preferably, the lumen pressure is maintained in a range of about 1 Pa to 10 Pa. More preferably, the lumen pressure is about 5 Pa.

As described above, the combination of the microwave radiation and the applied static magnetic field cause the formation of an ECR plasma within each tube in the ECR zone. The tubes can be drawn through the ECR zone at a rate chosen to permit exposure of each section of the lumen to the ECR plasma for a selected time period, e.g., a few seconds, to treat the lumen. Various devices known in the art can be utilized to draw the tubes through the ECR zone, including reels, pulleys and other mechanical conveyor systems.

An apparatus similar to the apparatus 18 can be utilized to treat the external walls of the tubes 26 by exposing the walls to an ECR-generated plasma. In such an apparatus, the internal portion of each tube is either pressurized or evacuated so as to inhibit formation of an internal plasma. The pressure of gas outside of the tube is selected to be in a range (e.g., 0.1 to 1000 Pa) so as to permit creation of an ECR plasma in the vicinity of a section of the external wall residing in the ECR zone.

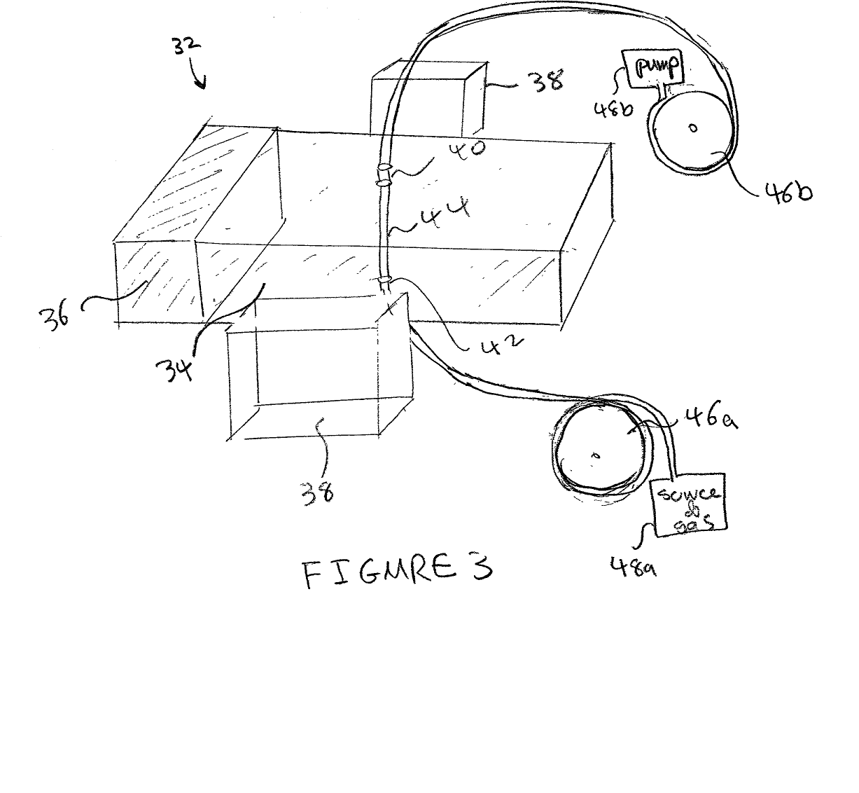

FIG. 3 illustrates an alternative apparatus 32 for implementing the methods of the invention. The apparatus 32 includes a waveguide 34 coupled to a source of microwave radiation 36, and a permanent magnet 38 positioned relative to the waveguide 34 so as to provide a static magnetic field within a portion of the waveguide to be utilized as an ECR zone. The magnetic field vector is preferably substantially perpendicular to the polarization of the microwave radiation traveling through the waveguide.

Two ports 40 and 42, which preferably include seals, e.g. lip seals, permit placing a section of a tube 44 within the portion of the waveguide 34 in which the magnetic field is applied. Two pulleys 46a and 46b can be utilized for passing different portions of the tube 44 through the waveguide 34 at a uniform or non-uniform speed. Further, a source of gas 48a and a pump 48b permit flowing a selected gas, e.g., argon, through the tube 44. The gas pressure within the section of the tube residing in the waveguide is selected to be in a range that allows initiating and maintaining a plasma within the tube. Further, the frequency of the microwave field and the amplitude of the static magnetic field are selected such that they substantially satisfy Equation (1).

When the gas within the tube is exposed to the microwave radiation in the presence of the permanent magnetic field, an ECR plasma is generated in a manner described above. The exposure of the inner surface of the tube to this plasma for a selected period treats the surface.

Although in this exemplary embodiment only one tubing section is shown, those skilled in the art will appreciate that the apparatus 32 can be utilized to treat simultaneously the surfaces of a plurality of tubular articles, e.g., catheters, by exposing them to an ECR-generated plasma.

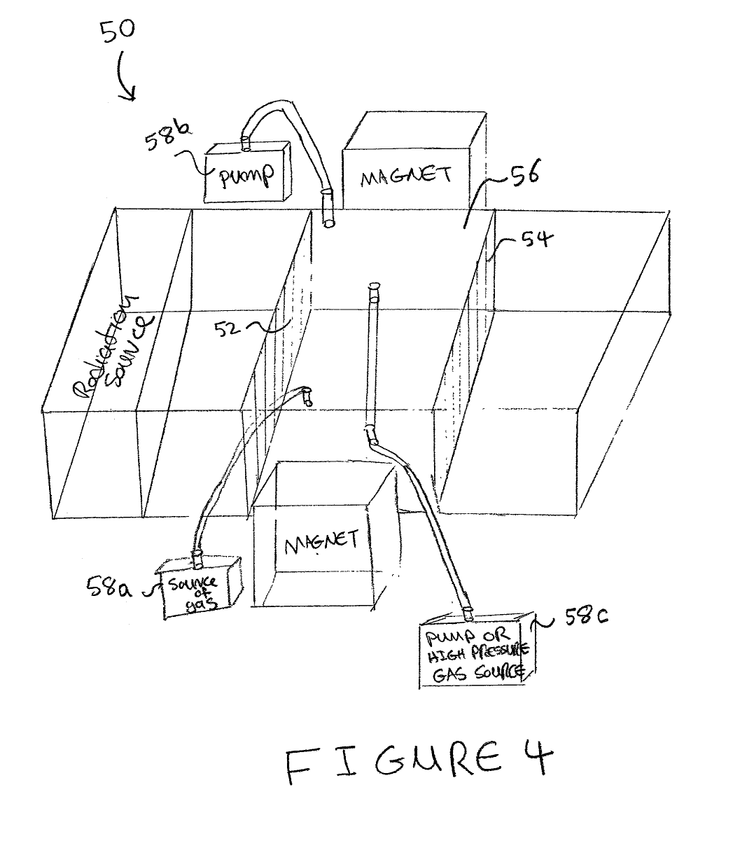

The apparatus 32 can be readily modified to allow treating the external surface of the tube 42 by exposing it to an ECR plasma. For example, FIG. 4 shows an apparatus 50, which is a modified version of the apparatus 32 of FIG. 3, in which two windows 52 and 54, which are substantially transparent to the radiation provided by the source 36, are positioned within the waveguide 34 so as to provide a gas enclosure 56. Each window 52/54 is preferably formed of a material having a low dielectric constant (e.g., a dielectric constant of about 1) at the frequency of the selected microwave radiation to minimize reflections, and thus enhance coupling of the radiation into the gas enclosure 56.

A source of gas 58a supplies a selected gas, e.g., argon, to the gas enclosure 56. The gas enclosure 56 is connected to a pump 58b which ensures that the pressure of the gas in the enclosure 56 is maintained in a range of approximately 0.1 to 1000 Pa, and more preferably in a range of approximately 1-10 Pa. Further, the tube 44 is connected to a pump or a source of high pressure gas 58c that either evacuates the lumen of the tube to provide a pressure therein that is sufficiently low to inhibit formation of a plasma in the lumen of the tube, or alternatively pressurizes the inner tube so as to prevent initiation of gas breakdown therein. The gas pressure in the enclosure 56, however, allows breakdown of the gas and formation of an ECR plasma. Thus, the external surface of the tube 44 can be treated by an ECR-generated plasma without affecting its internal wall.

Similar to the apparatus 32, the apparatus 50 can also be utilized to simultaneously treat the surfaces of a plurality of tubular articles, e.g., catheters.

It should be understood that the above embodiments of an apparatus for implementing the method of the invention are exemplary and are not intended to limit the scope of the invention. In particular, various modifications can be made to these embodiments without departing from the scope of the invention. For example, different radiation sources, radiation frequencies, and magnetic field amplitudes can be utilized to practice the invention.

What is claimed is:

1. A method of treating a surface of tubular article, the method comprising the steps of:
generating a gaseous plasma within a spatially-localized region of space by electron cyclotron resonance, and
exposing at least a portion of a surface of the tubular article to said plasma for a selected time period to treat the surface.

2. The method of claim 1, wherein said surface is selected to be at least a portion of the lumen of the tubular article.

3. The method of claim 1, wherein said surface is selected to be an outer surface of the tubular article.

4. The method of claim 1, wherein the step of generating the gaseous plasma includes
providing a magnetic field having a selected strength within said region of space, and
irradiating a gas contained within or flowing through said region of space with electromagnetic radiation having a frequency substantially equal to electron cyclotron frequency at said magnetic field strength so as to ionize the gas and produce said gaseous plasma.

5. The method of claim 4, further comprising the step of selecting a pressure of said gas to be in a range of about 0.1 Pa to about 1000 Pa.

6. The method of claim 5, further comprising selecting the gas pressure to be in a range of about 1 Pa to about 10 Pa.

7. The method of claim 6, further comprising selecting the gas pressure to be approximately 5 Pa.

8. The method of claim 4, wherein the frequency of the electromagnetic radiation (f_{c}) and the magnetic field amplitude (B) approximately satisfy the following equation:
$${f}_{c} = \frac{1}{2 π} e B / m$$
wherein e and m are the charge and mass of an electron, respectively.

9. The method of claim 4, wherein a ratio of the frequency of the electromagnetic radiation in units of Hertz and the magnetic field amplitude in units of Gauss is approximately 2.8×10⁶.

10. The method of claim 8, further comprising selecting the radiation frequency to be in a range of about 1 GHz to about 15 GHz.

11. The method of claim 10, further comprising selecting the amplitude of the magnetic field to be in a range of approximately 300 Gauss to 5500 Gauss.

12. The method of claim 4, further comprising selecting a power level of the electromagnetic radiation to be in a range of 10 to 500 Watts.

13. The method of claim 12, further comprising selecting the power level to be in a range of about 75 Watts to about 150 Watts.

14. The method of claim 13, further comprising selecting the power level to be approximately 100 Watts

15. The method of claim 2, further comprising the step of selecting an inner diameter of said tubular article to be in a range of about 0.5 mm to about 20 mm.

16. The method of claim 1, further comprising the step of drawing said tubular article through said region of space so as to expose different portions of the tubular surface to said plasma.

17. The method of claim 16, further comprising selecting a uniform speed for drawing said tubular article so as to provide a substantially uniform treatment of selected portions of the surface of the tubular article.

18. The method of claim 17, further comprising selecting a non-uniform speed for drawing said tubular article so as to effect a graded treatment of selected portions of the surface of the tubular article.

19. The method of claim 1, wherein said tubing is formed from the group consisting of electrically non-conductive organic polymer, and electrically non-conductive glass.

20. The method of claim 1, further comprising the step of coating said treated surface with a selected material.

21. The method of claim 20, wherein said coating material is selected to from the group consisting of an organic polymer, an inorganic material, and a bioactive material.

22. The method of claim 20, wherein said coating material is selected to be any of anti-thrombogenic, anti-coagulant, anti-biotic or anti-microbial.

23. The method of claim 20, wherein said anti-coagulant material is selected to be heparin.

24. The method of claim 20, wherein said coating material is selected to include any of one or more proteins, one or more vitamins, one or more minerals, or one or more enzymes.

25. The method of claim 20, wherein said coating material is selected to have anti-inflammatory analgesic properties.

26. The method of claim 20, wherein said coating material is selected to have cell growth properties.

27. The method of claim 4, further comprising selecting the gas from the group consisting of noble gases, diatomic gases, hydrocarbons, and fluorinated hydrocarbons.

28. The method of claim 27, wherein the gas can be selected to be any of argon, oxygen, nitrogen, methane, butane, and tetrafluoromethane.

29. The method of claim 4, further comprising selecting the gas such that said gaseous plasma effects direct deposition of a selected material from the plasma onto the surface of the tubular article.

30. The method of claim 29, wherein said gas is selected to be any of hydrocarbon or fluorinated hydrocarbon and said deposited material includes carbon as at least one constituent.

31. The method of claim 3, wherein the exposure of the outer surface to the plasma effects any of smoothing, sealing, reducing friction, sterilizing, or bond scission of the surface.

32. The method of claim 2, wherein the exposure of the portion of the lumen to the plasma effects any of smoothing, sealing, reducing friction, sterilizing, or bond scission of the lumen.

33. A method of treating a wall of a lumen, comprising the steps of:
placing a selected portion of said lumen in a treatment zone,
applying a magnetic field having a selected strength to the treatment zone,
introducing a gas into said lumen within said selected portion, said gas being in contact with the wall of said selected portion and having a selected partial pressure,
irradiating said gas with electromagnetic radiation having a frequency selected to be substantially equal to electron cyclotron frequency at said selected magnetic field strength so as to ionize said gas and create a plasma zone within said selected portion, said plasma treating said wall of the lumen.

34. The method of claim 33, wherein the strength of said magnetic field is selected to be in a range of approximately 300 Gauss to 5500 Gauss.

35. The method of claim 34, wherein the frequency of the electromagnetic radiation is selected to be in a range of about 1 GHz to 15 GHz.

36. The method of claim 33, further comprising exposing the selected portion of the lumen to the plasma for a pre-defined time period in order to effect a selected treatment of the lumen.

37. The method of claim 36, wherein the pre-defined time period is in a range from about one second to about one minute.

38. The method of claim 37, wherein the selected treatment is any of smoothing or sealing the lumen wall.

39. The method of claim 37, wherein the selected treatment effects any of reducing friction, sterilizing, or bond scission of the lumen wall.

40. An apparatus for treating a surface of a tubular article, comprising
a magnet for producing a static magnetic field within a region of space, said magnetic field having a selected amplitude,
a conduit adapted to couple to a source of gas to deliver a selected quantity of gas into at least a portion of said region of space in which the surface to be treated is placed, and
an electromagnetic-energy generator positioned so as to irradiate said region of space with electromagnetic radiation having a frequency substantially equal to an electron cyclotron frequency at said magnetic field magnitude so as to ionize said gas and create a plasma, said plasma treating the surface of the lumen.

41. The apparatus of claim 40, wherein said magnet is an electromagnet.

42. The apparatus of claim 40, wherein said electromagnetic-energy generator is a microwave oscillator.

43. The apparatus of claim 42, wherein said microwave oscillator is any of a magnetron or an IMPATT diode.

44. The apparatus of claim 42, further comprising a waveguide that transmits the electromagnetic radiation from the oscillator to the region of space.

45. The apparatus of claim 44, further comprising a horn attached to an end of said waveguide in proximity of said region of space, said horn providing an impedance match for efficient transmission of said radiation from said waveguide to said region of space.

46. The apparatus of claim 40, wherein said magnetic field amplitude is in a range of about 300 to about 5500 Gauss.

47. The apparatus of claim 46, wherein the radiation frequency is in a range of about 1 GHz to about 15 GHz.

48. An apparatus for treating a surface of a tubular article, comprising:
a magnet for producing a static magnetic field within a selected region of space forming a treatment zone, said magnetic field having a selected amplitude,
a waveguide section configured for connection to a source of microwave radiation, said waveguide section having two windows forming, in combination with the wall of the waveguide, an enclosure for storing a gas within the treatment zone, said windows being substantially transparent to the microwave radiation such that said source provides electromagnetic radiation within said enclosure having a selected frequency,
at least one port for allowing insertion of a portion of the tubular article into said gas enclosure,
wherein said radiation has a frequency substantially equal to electron resonance frequency at said selected amplitude of the magnetic field to ionize said gas and create a discharge within the treatment zone, said plasma being in contact with a selected surface of the tubular article to treat said surface.
